(19) United States
(12) Patent Application Publication
(10) Pub. No.: US 2021/0335504 A1
(43) Pub. Date: Oct. 28, 2021

(54) SYSTEM AND METHOD FOR COMPREHENSIVE EYE CARE

(71) Applicant: Abeer Ayoub, Clifton, NJ (US)

(72) Inventor: Abeer Ayoub, Clifton, NJ (US)

(21) Appl. No.: 17/370,786

(22) Filed: Jul. 8, 2021

Related U.S. Application Data
(60) Provisional application No. 63/131,650, filed on Dec. 29, 2020.

(51) Int. Cl. G16H 80/00; G16H 10/60; G16H 40/67; G16H 20/00

(52) U.S. Cl. CPC G16H 80/00; G16H 20/00; G16H 40/67; G16H 10/60

(57) ABSTRACT

A system and method for comprehensive eye care services including the services of ophthalmologists, Optometrists, opticians, eye exercise coach and trainers. The system allows for online eye consultation with eye care professionals, virtually try-on eyeglasses, and connect with opticians and pharmacies.

### FIELD OF THE INVENTION

The present invention relates to system and method for comprehensive eye care, and more particularly the present invention relates to a system and method for comprehensive eye care services.

### BACKGROUND

The eyecare industry is primarily concerned with the preservation of eye health and treatment of eye related problems. Eyecare is a large and growing healthcare industry in the United States and all over the world. Within the eyecare industry are professionals includes ophthalmologists who are medical doctors specialized in eye care, General physicians who are medical doctors, Optometrists who can test the vision, and opticians who generally sell prescription glasses and lenses.

Eyes are a vital part of the human body but because of the busy lifestyle, people often do not take needed heath care of their eyes. Thus, a long-standing need is there for an automated system for comprehensive eye care services for taking care of complete healthcare of an individual.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a system for comprehensive eye care services.

It is another object of the present invention that the system can schedule appointments for eye consultation.

It is still another object of the present invention that the system allows selecting the prescription glasses or sunglasses online.

It is yet a further object of the present invention that the system allows virtual try-on of the eyeglasses.

It is a further object of the present invention that the system alerts about expired prescription eyeglasses or lenses.

It is still a further object of the present invention that the system provides for renewing expired prescription eyeglasses or lenses.

It is yet another object of the present invention that the system provides for home eye consultations.

It is an additional object of the present invention that the system saves time and money of customers availing eye care services.

In one aspect, disclosed is a system and method for comprehensive eye care services including ophthalmologists, Optometrists, opticians, eye exercise coach, and trainers. The disclosed system provides a plurality of interfaces for eye care services.

In one aspect, the disclosed system may provide an interface for online eye consultation with ophthalmologists including Medical Groups and Private Practitioners. The interface may also provide for appointments in Hospitals and Nursing Homes. The interface may also aid in emergency eyecare.

In one aspect, the interface provides online eye consultation with ophthalmologists, Optometrists, opticians, eye exercise coaches and trainers. The interface can receive a prescription from the ophthalmologists or Optometrists and share the same with the opticians.

In one aspect, at least one interface allows a user to virtually try-on the eyeglasses, wherein a photograph of the face of the person can be received by at least one interface and 3D models of the one or more eyeglasses can be virtually superimposed on a 3D model of the face.

In one aspect, at least one interface can receive a request for home delivery of the prescription glasses or lens.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is an environment diagram showing the system connected to a user device and an ophthalmologist device, according to an exemplary embodiment of the present invention.

FIG. 2 is a block diagram showing the system having the interface module, teleconsultation module, optician module, and a profile module, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

Disclosed is a system and method for comprehensive eye care services including online eye consultation. The disclosed system saves valuable time and cost of eyecare consultation and optician visits. The disclosed system provides a range of eye care services online within a click away for the patients and customers of Pharmacies, Medical Groups, Private Practitioners, Hospitals, Urgent Care, Nursing Homes, and eyecare centers. The discloses system can connect to the customers, patients, and individuals through personal computing devices, such as desktop computers, workstations, smartphones, tablet computers, laptops, and like. The disclosed system can also connect with the customers, patients, and individuals through kiosks and safe booths in airports, malls, education institutes, and public areas.

A patient can get eye consultations online by registering with the disclosed system, through video conferencing with the ophthalmologists, opticians, eye care trainers, and like. The patient can also provide their medical history by uploading reports and diagnostic scans. Moreover, the patient's current and past medical history can be pulled from the electronic medical records.

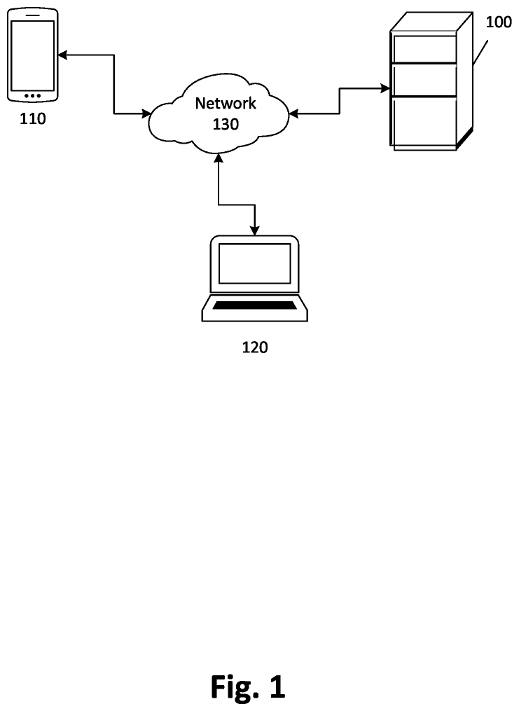

Referring to FIG. 1, which shows the disclosed system 100 can connect to the user device 110 and an eye care professional's device 120 through a network 130. The user device 110 and the eye care professional's device 120 can be computing devices having at least a display, microphone, speaker, and camera. External microphones, speakers, and a camera can also be connected to the computing device. FIG. 1 shows the user device as a smartphone and the eye care professional's device 120 as a laptop computer. Smartphones and laptop computers generally have the network circuitry for connecting to a network, such as the Internet.

The network 130 can be wired or wireless. For example, system 100 can be connected to the user device 110 through a wire. The network 130 can be a cellular communication network, LTE, UMTS, WiMAX, EV-DO, CDMA, GSM, Bluetooth, Bluetooth Low Energy, Bluetooth Smart, Wireless Fidelity (Wi-Fi), Zigbee, ANT, ANT+, Thread, Internet Protocol (IP), Ethernet, Fast Ethernet, Gigabit Ethernet, ATM, FDDI, Near Field Communication (NFC), or any other network known to a skilled person for sharing data packets between two electronic devices.

A patient can consult an eye care professional through videoconferencing, wherein the disclosed system can connect the user to a range of eye care professionals. For example, a patient having an eye infection, due to a busy schedule cannot visit a doctor. She using her smartphone can request the disclosed system for an online eye consultation. The disclosed system can connect the user device to one of the available eye care professionals. Alternatively, the user can choose from the available eye care professionals. The system can receive the symptoms for pre-screening and suggesting a doctor accordingly based on the pre-screening. The system can securely receive a prescription from the doctor, wherein the prescription can be presented on the user device and optionally recorded in the patient's electronic medical records. The patient can also request delivery of the prescribed medicine to his current location, wherein the current location of the patient can be shared with the system 100. The system 100, as shown in FIG. 1, can also be connected to a network of opticians and pharmacies. The disclosed system can securely share the prescription with a nearby pharmacy which can then deliver the medicine to the current address of the patient.

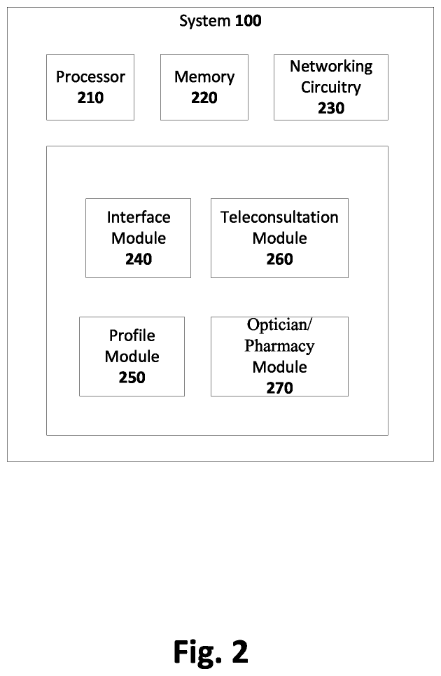

Referring to FIG. 2, which shows a block diagram of the disclosed system 100 having a processor 210, a memory 220 operably coupled to the processor, and a network circuitry 230. The memory includes an interface module 240, a profile module 250, a teleconsultation module 260, and an optician/pharmacy module 270. The interface module 240, upon execution by the processor, provides multiple digital interfaces on a display of electronic devices for users to interact with the disclosed system and for videoconferencing. The profile module 250, upon execution by the processor, can receive details of a user include contact details, prescription details, and medical records, and like related to the heath of the eye. The teleconsultation module 260, upon execution by the processor, can provide online consultation of the user with eye care professionals. The optician/pharmacy module 270, upon execution by the processor, can provide virtual try-on of eyeglasses and home delivery of prescription eyeglasses and lenses.

The interface module may provide multiple interfaces for the users to interact with the disclosed system. The interface can be provided as an application software on the user devices that allows the user device to communicate with the disclosed system. The application software can be developed for different platforms, such as Windows, Android, and iOS. Alternatively, one or more interfaces can be provided as a web interface that can be accessed on a user device through web browsers.

The profile module 250 can receive the contact details of a user willing to use the features of the disclosed system. The contact details can include email ID and contact phone number. The profile module may also store the address of the user. The profile module may also allow a user to upload their medical records including the eyesight. The profile module can also track and show transactions of the user with the disclosed system, such as consultations, payment, orders, prescriptions, and much more.

The teleconsultation module 260 can provide for online eye consultation. The discloses system can allow eye care professionals to connect with the disclosed system for providing their services online through the disclosed system. The eye care professionals can create their profile which includes details like the specialization of the eyecare professional, experience, addresses for in-person visits, working hours, and other details. The eye care professional can also provide their fee, which can be the fee for each consultation or time-based fee. The disclosed system can maintain a database of the eyecare professional which can be queried based on the requirement of a patient. The system can query the available eye care professionals at a given time based on the working hours of the eye care professional. The teleconsultation module 260 can present an interface to a patient for receiving the query. For example, a patient can download an interface on his smartphone and request online consultation with a doctor. The patient may also provide more details, such as symptoms. For example, the user may provide an input “having redness and pain in the eye”. Based on the request and optionally the input, the disclosed system can query the database to retrieve one or more doctors. A list of one or more doctors with details like experience, reviews, and fees can be displayed to the patient. The patient can select the doctor, wherein the selected doctor can join a videoconference interface provided by the interface module 240. The patent can then consult the doctor online through videoconferencing, thus saving travel time. The teleconsultation module 260 can also automatically connect to the first available doctor based on the symptoms and profile of the patient. The teleconsultation module 260 may also provide an interface for the eye care professional to write prescriptions digitally. Alternatively, the eye care professional can also upload the prescription. The teleconsultation module 260 may also refer the patient to a doctor for an in-person visit, for example, a near-by doctor based on the current location of the patient. The disclosed system can book an appointment and receive the payment for the same.

The optician/pharmacy module 270 may allow a user to virtually try-on eyeglasses.

The disclosed system 100 through the interface module 240 may provide an interface through which a user can upload photographs or videos of their face to create a 3D model. Thereafter, the user can navigate through the available eyeglasses online, and can virtually try-on any eyeglasses on the 3D model of the face. 3D models of the eyeglasses can be stored in the disclosed system which can be superimposed on the 3D model of the face. This allows a user to buy eyeglasses online confidently and saving travel time. The optician/pharmacy module 270 may also provide for renewing the expired prescription glasses and lens of a user. Record of such prescription glasses and lens can be tracked by the profile module which can alert the user about the expiry date and renew options available. The optician/pharmacy module 270 can also provide for home-testing of eyes by an Optometrist or refer the user to a nearby Optometrist. The disclosed optician/pharmacy module 270 can also deliver the prescribed eyeglasses and medicines to the address of the user or the current location of the user. The optician/pharmacy module 270 can include a database of the opticians and pharmacies with their location coordinates. The optician/pharmacy module 270 can locate a nearby optician or pharmacy based on the address or current location of the user.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for providing comprehensive eye care, the method comprising the steps of:
providing a first digital interface on a user device to receive a request for online consultation from a patient;
retrieving records of one or more eye care professionals from a database based on the request;
presenting the records of the one or more eye care professionals on the first digital interface;
receiving, a selection of at least one eye care professional from the records of the one or more eye care professionals; and
presenting, a second digital interface, on at least one eye care professional's device, the second digital interface permits the at least one eye care professional to communicate with the patient through videoconferencing.

2. The method according to claim 1, wherein the method further comprises the step of:
receiving, by the second digital interface, a prescription from the at least one eye care professional.

3. The method according to claim 2, wherein the method further comprises the step of:
presenting, the prescription on a third digital interface, the third digital interface downloaded on a pharmacy/optician's device.

4. The method according to claim 3, wherein the method further comprises the step of:
receiving, a current location of the patient; and
locating the pharmacy/optician's device nearby the current location of the patient.

5. The method according to claim 1, wherein the method further comprises the step of:
receiving, through the first digital interface, photographs of a face of the patient;
creating a 3D model of the face; and
superimposing a 3D model of an eyeglass on the 3D model of the face.

6. The method according to claim 1, wherein the first digital interface can be configured on a display of a public kiosk.

7. The method according to claim 1, wherein the first digital interface can be configured on a display of a smartphone.
